(19) United States
(12) Patent Application Publication
(10) Pub. No.: US 2021/0346657 A1
(43) Pub. Date: Nov. 11, 2021

(54) MULTIFUNCTIONAL ANGIOPLASTY AND ANGIOGRAPHY CATHETER CONVERTIBLE BETWEEN OVER-THE-WIRE AND RAPID-EXCHANGE CONFIGURATIONS AND METHODS OF USE THEREOF

(71) Applicant: Timothy Murphy, Providence, RI (US)

(72) Inventor: Timothy Murphy, Providence, RI (US)

(21) Appl. No.: 16/866,907

(22) Filed: May 5, 2020

(51) Int. Cl. A61M 25/10; A61M 25/00

(52) U.S. Cl. CPC A61M 25/104; A61M 25/0026; A61M 2025/0183; A61M 2025/1063; A61M 25/10181

(57) ABSTRACT

Disclosed is a multifunctional convertible catheter and methods of use, said multifunctional convertible catheter capable of performance of both diagnostic angiography while containing a guide wire throughout its lumen and angioplasty, and also being convertible between over-the-wire or rapid exchange configurations by manipulation of said multifunctional catheter and said guide wire by an operator. Whether used in an over-the-wire or rapid exchange configuration, a multifunctional convertible catheter disclosed herein allows the operator to perform angioplasty at any site in the body without use of an exchange length guide wire, without the need to exchange said convertible angioplasty balloon catheter for a diagnostic catheter pre- or post-angioplasty, without the need of an injectable vascular sheath, all while reducing catheter inventory requirements for the health care system.

### CROSS-REFERENCE TO RELATED APPLICATIONS

This application claims priority under 35 U.S.C. § 119 to U.S. Provisional App. Nos. 62/865,154, filed 22 Jun. 2019, entitled “Angioplasty Balloon Catheter Convertible Between Over-The-Wire and Rapid-Exchange Configurations”, 62/865,153, filed 22 Jun. 2018, entitled “Angioplasty Balloon Catheter Convertible Between Over-The-Wire and Rapid-Exchange Configurations”, and 62/865,152, filed 22 Jun. 2019, entitled “Angioplasty Balloon Catheter Convertible Between Over-The-Wire and Rapid-Exchange Configurations”, the entireties of which are incorporated by reference herein.

### FIELD OF THE INVENTION

The present invention relates to medical devices and vascular interventional methods. More particularly, the present invention is directed to devices used for the diagnosis and angioplasty of intravascular stenoses, and methods of use thereof.

### BACKGROUND

### U.S. Patents

| Pat. No. | Issue Date | Patentee |
|---|---|---|
| U.S. Pat. No. 10,149,962 | Dec. 11, 2018 | Franklin |
| U.S. Pat. No. 8,532,749 | Sep. 10, 2013 | Patton |
| U.S. Pat. No. 7,873,404 | Jan. 18, 2011 | Patton |
| U.S. Pat. No. 6,402,720 | Jun. 11, 2002 | Miller |
| U.S. Pat. No. 6,322,577 | Nov. 27, 2001 | McInnes |
| U.S. Pat. No. 6,007,517 | Dec. 28, 1999 | Anderson |
| U.S. Pat. No. 5,538,510 | Jul. 23, 1996 | Fontirroche |
| U.S. Pat. No. 5,489,271 | Feb. 6, 1996 | Anderson |
| U.S. Pat. No. 5,484,412 | Jan. 16, 1996 | Pierpont |
| U.S. Pat. No. 5,484,411 | Jan. 16, 1996 | Inderbitzen et al. |
| U.S. Pat. No. 5,472,425 | Apr. 22, 1994 | Teirstein |
| U.S. Pat. No. 5,472,425 | Dec. 5, 1995 | Teirstein |
| U.S. Pat. No. 5,470,314 | Nov. 28, 1995 | Walinsky |
| U.S. Pat. No. 5,433,706 | Jul. 18, 1995 | Abiuso |
| U.S. Pat. No. 5,425,714 | Jun. 20, 1995 | Johnson et al. |
| U.S. Pat. No. 5,425,709 | Jun. 20, 1995 | Gambale |
| U.S. Pat. No. 5,411,478 | May 2, 1995 | Stillabower |
| U.S. Pat. No. 5,409,458 | Apr. 25, 1995 | Khairkhahan et al. |
| U.S. Pat. No. 5,403,274 | Apr. 4, 1995 | Cannon |
| U.S. Pat. No. 5,395,353 | Mar. 7, 1995 | Scribner |
| U.S. Pat. No. 5,395,333 | Mar. 7, 1995 | Brill |
| U.S. Pat. No. 5,383,890 | Jan. 24, 1995 | Miraki et al. |
| U.S. Pat. No. 5,383,856 | Jan. 24, 1995 | Bersin |
| U.S. Pat. No. 5,383,853 | Jan. 24, 1995 | Jung |
| U.S. Pat. No. 5,378,237 | Jan. 3, 1995 | Boussignac et al. |
| U.S. Pat. No. 5,370,617 | Dec. 6, 1994 | Sahota |
| U.S. Pat. No. 5,346,505 | Sep. 13, 1994 | Leopold |
| U.S. Pat. No. 5,342,297 | Aug. 30, 1994 | Jang |
| U.S. Pat. No. 5,336,184 | Aug. 9, 1994 | Teirstein |
| U.S. Pat. No. 5,336,184 | Aug. 9, 1994 | Teirstein |
| U.S. Pat. No. 5,328,469 | Jul. 12, 1994 | Coletti |
| U.S. Pat. No. 5,324,269 | Jun. 28, 1994 | Miraki |
| U.S. Pat. No. 5,318,535 | Jun. 7, 1994 | Miraki |
| U.S. Pat. No. 5,308,356 | May 3, 1994 | Blackshear, Jr. et al. |
| U.S. Pat. No. 5,295,995 | Mar. 22, 1994 | Kleiman |
| U.S. Pat. No. 5,295,959 | Mar. 22, 1994 | Gurbel et al. |
| U.S. Pat. No. 5,284,473 | Feb. 8, 1994 | Calabria |
| U.S. Pat. No. 5,267,958 | Dec. 7, 1993 | Buchbinder et al. |
| U.S. Pat. No. 5,232,445 | Aug. 3, 1993 | Bonzel |
| U.S. Pat. No. 5,205,822 | Apr. 27, 1993 | Johnson et al. |
| U.S. Pat. No. 5,180,367 | Jan. 19, 1993 | Kontos et al. |
| U.S. Pat. No. 5,171,222 | Dec. 15, 1992 | Euteneuer et al. |
| U.S. Pat. No. 5,158,540 | Oct. 27, 1992 | Wijay et al. |
| U.S. Pat. No. 5,156,594 | Oct. 20, 1992 | Keith |
| U.S. Pat. No. 5,156,594 | Oct. 20, 1992 | Keith |
| U.S. Pat. No. 5,154,725 | Oct. 13, 1992 | Leopold |
| U.S. Pat. No. 5,147,377 | Sep. 15, 1992 | Sahota |
| U.S. Pat. No. 5,135,535 | Aug. 4, 1992 | Kramer |
| U.S. Pat. No. 5,116,318 | May 26, 1992 | Hillstead |
| U.S. Pat. No. 5,108,370 | Apr. 28, 1992 | Walinsky |
| U.S. Pat. No. 5,102,403 | Apr. 7, 1992 | Alt |
| U.S. Pat. No. 5,087,247 | Feb. 11, 1992 | Horn |
| U.S. Pat. No. 5,078,685 | Jan. 7, 1992 | Colliver |
| U.S. Pat. No. 5,061,273 | Oct. 29, 1991 | Yock |
| U.S. Pat. No. 5,061,273 | Oct. 29, 1991 | Yock |
| U.S. Pat. No. 5,049,131 | Sep. 17, 1991 | Deuss |
| U.S. Pat. No. 5,046,503 | Sep. 10, 1991 | Schneiderman |
| U.S. Pat. No. 5,046,497 | Sep. 10, 1991 | Millar |
| U.S. Pat. No. 5,040,548 | Aug. 20, 1991 | Yock |
| U.S. Pat. No. 5,040,548 | Aug. 20, 1991 | Yock |
| U.S. Pat. No. 4,994,033 | Feb. 19, 1991 | Shockey et al. |
| U.S. Pat. No. 4,988,356 | Jan. 29, 1991 | Crittenden |
| U.S. Pat. No. 4,988,356 | Jan. 29, 1991 | Crittenden et al. |
| U.S. Pat. No. 4,983,167 | Jan. 8, 1991 | Sahota |
| U.S. Pat. No. 4,944,745 | Jul. 31, 1990 | Sogard et al. |
| U.S. Pat. No. 4,909,781 | Mar. 20, 1990 | Husted |
| U.S. Pat. No. 4,909,252 | Mar. 20, 1990 | Goldberger |
| U.S. Pat. No. 4,850,969 | Jul. 25, 1989 | Jackson |
| U.S. Pat. No. 4,842,590 | Jun. 27, 1989 | Tanabe |
| U.S. Pat. No. 4,824,435 | Apr. 25, 1989 | Giesy |
| U.S. Pat. No. 4,819,751 | Apr. 11, 1989 | Shimada et al. |
| U.S. Pat. No. 4,790,315 | Dec. 13, 1988 | Mueller, Jr. et al. |
| U.S. Pat. No. 4,771,777 | Sep. 20, 1988 | Horzewski |
| U.S. Pat. No. 4,762,129 | Aug. 9, 1988 | Bonzel |
| U.S. Pat. No. 4,762,129 | Aug. 9, 1988 | Bonzel |
| U.S. Pat. No. 4,748,982 | Jun. 7, 1988 | Horzewski |
| U.S. Pat. No. 4,641,654 | Feb. 10, 1987 | Samson |
| U.S. Pat. No. 4,601,713 | Jul. 22, 1986 | Fuqua |
| U.S. Pat. No. 4,581,017 | Apr. 8, 1986 | Sahota |
| U.S. Pat. No. 4,581,017 | April, 1986 | Sahota |
| U.S. Pat. No. 4,323,071 | Apr. 6, 1982 | Simpson |
| U.S. Pat. No. 4,195,637 | Apr. 1, 1980 | Gruntzig |
| U.S. Pat. No. 3,382,872 | May 14, 1968 | Rubin |

Foreign Patent Application

German patent application P 39 34 695.1 Rupprecht

### PUBLICATIONS

Nordenstrom, B. Balloon catheters for percutaneous insertion into the vascular system. Acta Radiol 1962; 57:411-416.
Nordenstrom, B. New instruments for catheterization and angiocardiography. Radiology 1965; 85:256-259.

This description of art is not intended to constitute an admission that any patent, publication or other information referred to is “prior art” with respect to this invention, unless specifically designated as such. In addition, this section should not be construed to mean that a search has been made or that no other pertinent information as defined in 37 C.F.R. § 1.56 (a) exists.

Angioplasty balloon catheters have been used to dilate blockages in blood vessels for over 40 years. The original angioplasty balloon catheter as used by Gruentzig in 1977 was a fixed-wire platform. Subsequently, Simpson and Robert developed a movable guide wire balloon platform, or “over-the-wire” (“OTW”), U.S. Pat. No. 4,323,071. The OTW configuration includes a lumen used to inflate the angioplasty balloon and usually a second lumen used for passage of a guide wire therethrough. In the OTW configuration, the guide wire exit port is usually at the distal tip of the catheter, and the guide wire entry port is at the hub or proximal end of the catheter.

In 1984 Bonzel introduced the concept of the “rapid exchange” (“RX”), or monorail, configuration, U.S. Pat. No. 4,762,129. With the RX configuration, the guide wire exit port is at the distal tip of the catheter, but the guide wire entry port is substantially distal to the hub or proximal end of the catheter and proximal to an angioplasty balloon.

### SUMMARY

The present invention comprises a multifunctional convertible catheter which performs both diagnostic angiography even when containing a guide wire, and which can be converted in real time from “over-the-wire” to “rapid-exchange” configuration. Said multifunctional convertible catheter comprises a a proximal end and a distal end, on which at least one or more hub adapters is disposed toward said proximal end, and further on which an angioplasty balloon is disposed toward a distal end of said angioplasty balloon catheter, said multifunctional convertible catheter having a plurality of lumens including at least a lumen for inflation and deflation of said angioplasty balloon, and at least a second lumen for slidable movement of a guide wire therethrough, said second lumen extending from said proximal end through a length of said multifunctional convertible catheter to said distal end, said second lumen having at least a proximal guide wire port at said proximal end and a distal guide wire port at said distal end of said multifunctional convertible catheter, said second lumen also comprising at least a third intermediate port disposed along said multifunctional convertible catheter shaft distal to said proximal end and proximal to said angioplasty balloon, and further, said second lumen allowing passage of fluids admitted through said proximal guide wire port and exiting through said third intermediate port proximal to said angioplasty balloon even while a guide wire remains in position throughout a length of said guide wire lumen. Furthermore, the invention disclosed herein comprises a multifunctional convertible catheter that is capable of being used convertibly either in an OTW configuration or in an RX configuration.

In order for a multifunctional convertible catheter disclosed herein to be used in an OTW configuration, the operator gains access to the vascular system using the Seldinger technique whereby a guide wire is placed through the skin, into a blood vessel, and directed into a vascular territory to be treated. Once other devices such as a needle or a catheter is removed, a multifunctional convertible catheter can be “backloaded” (i.e., passed over the proximal end of the guide wire) and advanced into the body to the vascular territory to be treated. Another way of introducing a multifunctional convertible catheter into the body would be to place a vascular access sheath over a guide wire in situ and then introduce said multifunctional convertible catheter over a guide wire into said vascular sheath and thereby into a body. Without further manipulation, said guide wire when used in this way may eventually pass through the length of the convertible angioplasty balloon catheter and exit from its proximal end.

Alternatively, if the operator so choses, the multifunctional convertible catheter can be used in the RX configuration by passing said multifunctional convertible catheter over a guidewire until said guide wire is visible in said third intermediate port, wherein an operator can manipulate said multifunctional convertible catheter and said guide wire until said guide wire exits said third intermediate port, achieving said RX configuration

One familiar with the art will recognize that by controlling a caliber of said second lumen proximal to said third intermediate port, said second lumen distal to said third intermediate port, and the size of said third intermediate port, fluids can be injected around said guide wire when used in the OTW configuration and exit said third port, which an operator could use to advantage for example for the injection of radiopaque contrast material, flush solutions, or medications. Similarly, when used in the RX configuration an operator could use said second lumen for injection of fluids which would exit said multifunctional convertible catheter from said third port.

### BRIEF DESCRIPTION OF THE DRAWINGS

FIG. 1 shows an exemplary embodiment of the invention comprising a multifunctional convertible catheter with a guide wire in OTW configuration, longitudinal section view.

FIG. 2 illustrates an alternative embodiment of the invention comprising an exemplary embodiment of a multifunctional convertible catheter with a guide wire in RX configuration, longitudinal section view.

FIG. 3 shows an example of a multifunctional catheter, surface view.

### DETAILED DESCRIPTION

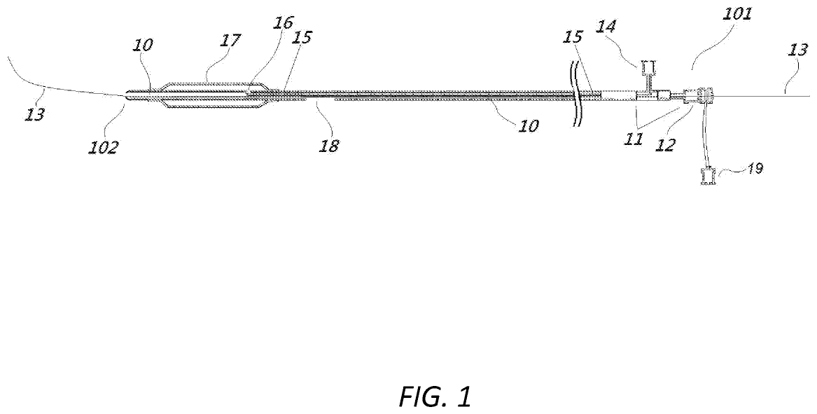

Referring now to FIG. 1, there is shown an exemplary embodiment of the present invention that comprises a multifunctional convertible catheter, said multifunctional convertible catheter having a proximal end 101 and a distal end 102, and an angioplasty balloon 17 disposed generally toward said distal end 102, said proximal end 101 also comprising a hub 11 that comprises a balloon inflation lumen adapter 14 and a guide wire lumen adapter 12, said balloon inflation lumen adapter 14 in continuous communication with a balloon inflation lumen 15, said balloon inflation lumen 15 further in continuous communication with a port 16 communicating with an inner aspect of said angioplasty balloon 17, said guide wire lumen adapter 12 in continuous communication with a guide wire lumen 10, said guide wire lumen 10 extending continuously from said proximal end 101 throughout an entire length of said multifunctional convertible catheter to said distal end 102, said guide wire lumen 10 also having a third intermediate port 18 disposed along its length proximal to said angioplasty balloon 17 and distal to said hub 11. In this exemplary embodiment, a guide wire 13 extends through an entire length of said guide wire lumen 10, and as such this embodiment illustrates OTW configuration of said multifunctional convertible catheter. In this exemplary embodiment, a side arm adapter 19 is reversibly attached to said guide wire lumen adapter 12 to enable injection of fluids there through and around said guide wire 13 while maintaining said guide wire 13 in position throughout said guide wire lumen 10 from said proximal end 101 to said distal end 102. In OTW configuration, appropriate sizing of said guide wire lumen 10 proximal and distal to said third intermediate port 18, fluids can be admitted through said guide wire lumen adapter 12, from which said fluids would flow continuously around said guide wire 13 within said guide wire lumen 10, and exit said third intermediate port 18. In order to permit injection of radiopaque contrast, fluids, or medications through said guide wire lumen 10 using any means including manual injection with a syringe said guide wire lumen 10 would be of sufficiently larger inner diameter to permit said injection even when a guide wire 13 is in place in said guide wire lumen 10. It can be appreciated that the distribution of said fluid flow exiting either said intermediate port 18 or said distal end 102 could be adjusted by appropriate sizing of said guide wire lumen 10 proximal to said third intermediate port 18, said guide wire lumen 10 distal to said third intermediate port 18, and an opening of third intermediate port 18.

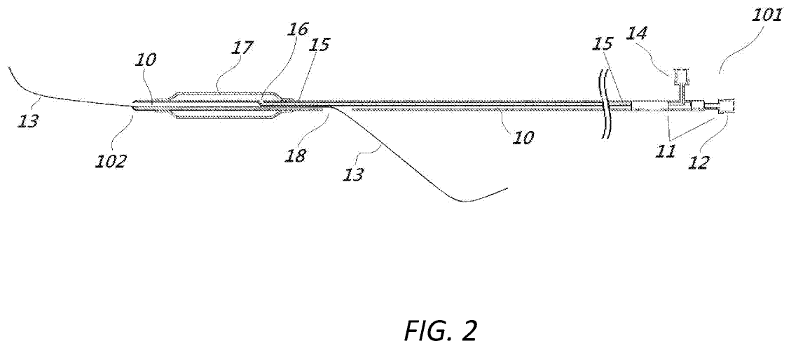

Turning now to FIG. 2, there is shown an exemplary embodiment of the present invention that comprises a multifunctional convertible catheter, said multifunctional convertible catheter having a proximal end 101 and a distal end 102, and an angioplasty balloon 17 disposed generally toward said distal end 102, said proximal end 101 also comprising a hub 11 that comprises a balloon inflation lumen adapter 14 and a guide wire lumen adapter 12, said balloon inflation lumen adapter 14 in continuous communication with a balloon inflation lumen 15, said balloon inflation lumen 15 further in continuous communication with a port 16 communicating with an inner aspect of said angioplasty balloon 17, said guide wire lumen adapter 12 in continuous communication with a guide wire lumen 10, said guide wire lumen 10 extending continuously from said proximal end 101 throughout an entire length of said multifunctional convertible catheter to said distal end 102, said guide wire lumen 10 also having a third intermediate port 18 disposed along its length distal to said proximal end 101 and proximal to said angioplasty balloon 17 and distal to said hub 11. In this exemplary embodiment, a guide wire 13 extends from a distal end 102 of said convertible angioplasty balloon catheter through a partial length of said guide wire lumen 10, and exits said third intermediate port 18, and as such this embodiment illustrates an RX configuration of said multifunctional convertible catheter. In said RX configuration, if said guide wire lumen 10 and said intermediate port 18 were sized appropriately, fluids could be injected through said guide wire lumen adapter 12, from which they would flow continuously within said guide wire lumen 10 and exit said intermediate port 18 even without said guide wire 13 in position there through. It can be appreciated that the distribution of said fluid flow exiting either said intermediate port 18 or said distal end 102 could be adjusted by appropriate sizing of said guide wire lumen 10 proximal to said third intermediate port 18, said guide wire lumen 10 distal to said third intermediate port 18, and an opening of third intermediate port 18.

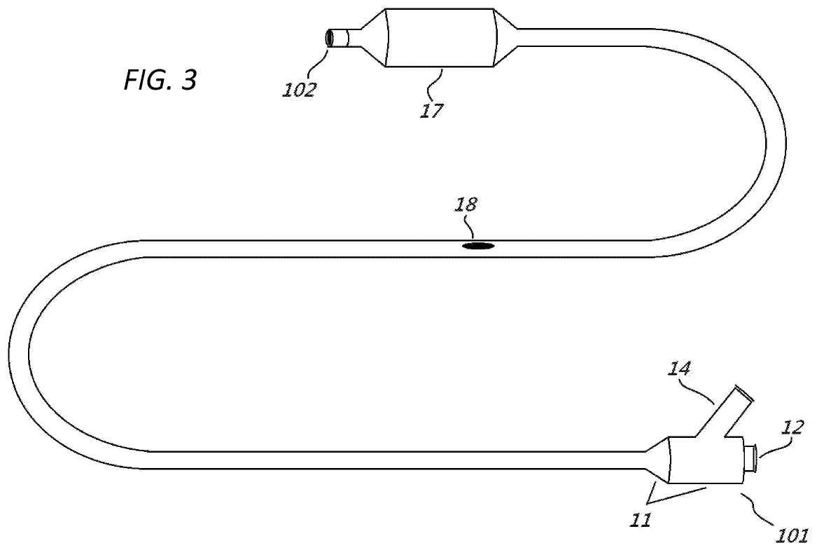

In FIG. 3, another exemplary embodiment of the invention is shown, comprising a multifunctional convertible catheter, with a proximal end 101 and a distal end 102, an angioplasty balloon 17 disposed generally toward a distal end 102 of said convertible angioplasty balloon catheter, a hub 11 disposed generally toward said proximal end 101 including at least an angioplasty balloon inflation lumen adapter 14 and at least a guide wire lumen adapter 12, and a third intermediate port 18 disposed along the length of said convertible angioplasty balloon catheter between said hub 11 and said angioplasty balloon 17.

There are several circumstances in which a multifunctional convertible catheter could be used to advantage. For example, a multifunctional convertible catheter could be used in its OTW configuration when suitable, such as when an angioplasty is performed either close to an access site where an exchange-length guide wire exchange is not required, or when a guide wire is pre-loaded into a convertible angioplasty balloon catheter and both introduced together, for example through a guide sheath. When an angioplasty is done in steps that include catheterization of the blood vessel to be treated with a diagnostic angiography catheter and guide wire, and when that guide wire is not of suitable exchange length, said diagnostic angiography catheter can be exchanged for said multifunctional convertible catheter used in an RX configuration over said guide wire. Another advantage of said multifunctional convertible catheter is in streamlining inventory. Since said multifunctional convertible catheter is capable of treating blood vessels that require OTW angioplasty balloons as well as RX angioplasty balloons, and capable of performing diagnostic arteriography and angioplasty with a single device, inventory needs will be reduced and costs to hospitals and health care systems will be reduced. Furthermore, when using a combination-type angioplasty balloon catheter that comprises an injection lumen with an injection lumen exit port along its length, such as disclosed in U.S. Pat. No. 8,532,749, an entire angioplasty procedure can be done with a single multifunctional convertible catheter as disclosed herein in the OTW configuration as follows:
gain vascular access in a patient using Seldinger's technique or other methods,
place a vascular access sheath in a vascular tree,
introduce a multifunctional convertible catheter with a guide wire preloaded in the OTW configuration into said vascular tree through said sheath, in one example with a shape on the guide wire to enable selective cannulation of a blood vessels of interest,
advance said multifunctional convertible catheter to an appropriate position within said blood vessel,
inject contrast into a guide wire lumen through a hub adapter 14, said contrast exiting said guide wire lumen primarily through a third intermediate port 18 along a shaft of said multifunctional convertible catheter, and obtaining radiographic angiogram images,
position said angioplasty balloon in a location within a blood vessel to be treated using fluoroscopic observation of injected radiopaque contrast material into said guide wire lumen 10,
dilate said blood vessel in an area to be treated using said angioplasty balloon 17,
deflate said angioplasty balloon and reposition said multifunctional convertible catheter to permit contrast injection to obtain suitable radiographic angiogram images,
inject contrast into a guide wire lumen 10 through a hub adapter 14, said contrast exiting said guide wire lumen primarily through an intermediate port 18 along a shaft of said multifunctional convertible catheter, and obtaining radiographic angiogram images, and
remove devices from said patient's body.

Another way to perform said angioplasty procedure would be to post-load said convertible angioplasty balloon catheter over said guide wire after placement of said guide wire using a selective diagnostic catheter, said guide wire being of sufficient length to accommodate said convertible angioplasty balloon catheter in OTW configuration, or having docking ability so that said guide wire can be extended in length to accommodate said convertible angioplasty balloon catheter in OTW configuration. Additionally, when using a combination-type angioplasty balloon catheter that comprises an injection lumen with an injection lumen exit port along its length, such as disclosed in U.S. Pat. No. 8,532,749, an entire angioplasty procedure could be done with a single multifunctional convertible catheter as disclosed herein in the RX configuration as follows:
gain vascular access in a patient using Seldinger's technique or other methods,
place a vascular access sheath in a vascular tree,
use a combination of a selective diagnostic catheter and guide wire to selective catheterize an artery of interest,
remove said diagnostic catheter while maintaining said guide wire in situ,
introduce said multifunctional convertible catheter by “back loading” over said guide wire 13,
as said guide wire 13 passes proximally within a guide wire lumen 10 of said multifunctional convertible catheter an operator can observe its entry into the catheter segment bounded by a third intermediate port 18, whereby said operator can manipulate said multifunctional convertible catheter and said guide wire such that said guide wire passes through said third intermediate port 18 and exterior to said multifunctional convertible catheter shaft,
advance said multifunctional convertible catheter to an appropriate position within a blood vessel to be treated over said guide wire 13,
inject contrast into a guide wire lumen 10 through a hub adapter 14, said contrast exiting a third intermediate port 18 along a shaft of said multifunctional convertible catheter, and obtain radiographic angiogram images,
position said multifunctional convertible catheter such that said angioplasty balloon 17 can perform angioplasty in a blood vessel to be treated using fluoroscopic observation of radiopaque contrast injected into said guide wire lumen 10,
dilate said blood vessel in area to be treated using said angioplasty balloon 17,
deflate said angioplasty balloon 17 and reposition said multifunctional convertible catheter to obtain radiographic angiogram images,
inject contrast into a guide wire lumen 10 through a hub adapter 14, said contrast exiting said guide wire lumen primarily through a third intermediate port 18 along a shaft of said multifunctional convertible catheter, and obtaining radiographic angiogram images, and
remove devices from said patient's body. Whether used in OTW or RX configuration, the multifunctional convertible catheter disclosed herein allows the operator to perform angioplasty at any site in the body without use of an exchange length guide wire, without the need to exchange said multifunctional convertible catheter for a diagnostic catheter pre- or post-angioplasty, without the need of use of a vascular sheath, all while reducing angioplasty balloon catheter and diagnostic catheter inventory requirements for the health care system.

It will be appreciated by those familiar with the art that an operator would make the determination of whether to use said multifunctional convertible catheter in an OTW or RX configuration flexibly and during performance of the procedure, based on procedural considerations such as for example the proximity of the treatment site, ease of access and guide wire placement, need to perform catheter exchanges, among other technical procedural considerations. It can further appreciated that some of these steps can be done using other sequences standard in the art, for example, using a diagnostic catheter to obtain angiogram images prior to exchanging for a multifunctional convertible catheter to perform angioplasty.

### INCORPORATION BY REFERENCE

References and citations to other documents, such as patents, patent applications, provisional patent applications, patent publications, journals, books, papers, web content, that have been made throughout this disclosure are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein. It is my intention that all such revisions and modifications as are obvious to those skilled in the art will be included within the scope of the following claims.

### CONCLUSIONS

The reader will see that a multifunctional convertible catheter of the various embodiments described herein offers numerous advantages, having the potential to incorporate the advantages of both OTW and RX systems as the circumstances indicate, providing flexibility to the operator.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiments were chosen and described in order to best explain the principles of the present invention and its practical application, to thereby enable others skilled in the art to best utilize the present invention and various embodiments with various modifications as are suited to the particular use contemplated.

What is claimed is:

1. In combination, a medical multifunctional convertible catheter for use in angioplasty procedures comprising:
(a) a catheter having a proximal end and a distal end,
(b) an expansile balloon securely mounted on said catheter generally disposed toward said distal end of said multifunctional convertible catheter,
(c) a plurality of lumens incorporated in said multifunctional convertible catheter including at least a first lumen as a means for passage of a guide wire and injection of fluids there through, said first lumen extending continuously from said proximal end to said distal end of said catheter, said first lumen further comprising at least a proximal port at said proximal end of said catheter, at least a distal port at said distal end of said catheter, and at least a third intermediate port located proximal to said expansile balloon and distal to said proximal end of said catheter, said intermediate port comprising an opening through a wall of said catheter as a means for passage of fluids and a guide wire there through, said multifunctional convertible catheter further comprising a means for injection of fluids into said first lumen with a guide wire in position throughout said first lumen from said proximal end to said distal end, and at least a second lumen as a means for injection of pressurized fluids or gases to inflate said expansile balloon.

2. A medical multifunctional convertible catheter of claim 1, wherein said proximal end comprises at least a first hub adapter at a proximal end of said first lumen, said first hub adapter further comprising a means for injection of fluids through said first lumen with a guide wire in position throughout said first lumen, and at least a second hub adapter at a proximal end of said second lumen.

3. A medical multifunctional convertible catheter of claim 1, wherein an operator can manually modify a configuration of said multifunctional convertible catheter such that said guide wire passes slidably through an entire length of said first lumen when said catheter is used in an “over-the-wire” (OTW) configuration or through only part of a length of said first lumen when used in a “rapid exchange” (RX) configuration.

4. A medical multifunctional convertible catheter of claim 1, wherein a guide wire passes slidably through a partial length of said first lumen when said catheter is used in a “rapid exchange” (RX) configuration, said partial length comprising a catheter segment between said distal port and said third intermediate port.

5. A medical angioplasty balloon catheter of claim 1, wherein said first lumen is of sufficient size that it permits adequate fluid flow therethrough when injected using conventional medical means, even when a guide wire is within said first lumen.

6. A medical angioplasty balloon catheter of claim 1, wherein said third intermediate port is a means of egress for contrast injected into said first lumen.

7. A balloon angioplasty catheter with a contrast injection port proximal to an angioplasty balloon that be converted between over-the-wire and rapid-exchange configurations.

8. A method of performing an angioplasty comprising use of multifunctional convertible catheter comprising a proximal end and a distal end, and said proximal end comprising at least a first hub adapter at a proximal end of said first lumen, and at least a second hub adapter at a proximal end of said second lumen, and at least an expansile balloon securely mounted on said catheter generally disposed toward said distal end of said catheter, said multifunctional convertible catheter comprising a plurality of lumens including at least a first lumen as a means for passage of a guide wire and injection of fluids there through, said first lumen extending continuously from said proximal end to said distal end of said catheter, said first lumen including at least a proximal port at said proximal end of said catheter, at least a distal port at said distal end of said catheter, and at least a third intermediate port located proximal to said expansile balloon of said catheter and distal to said proximal end of said catheter, said intermediate port comprising an opening through a wall of said catheter, said multifunctional convertible catheter further comprising a means for injection of fluids into said first lumen with a guide wire in position throughout said first lumen from said proximal end to said distal end, and said multifunctional convertible catheter further comprising at least a second lumen as a means for injection of pressurized fluids or gases to inflate said expansile balloon, said method of performing an angioplasty comprising an “over-the-wire” (OTW) angioplasty by gaining vascular access in a patient using Seldinger's technique or other methods, placing a vascular access sheath in a vascular tree, introducing a multifunctional convertible catheter with a guide wire preloaded in the OTW configuration into said vascular tree through said sheath, with a shape on the guide wire to enable selective cannulation of a blood vessels of interest, advancing said convertible angioplasty balloon catheter to an appropriate position within said blood vessel, injecting contrast into a guide wire lumen through a hub adapter, said contrast exiting said guide wire lumen primarily through a third intermediate port along a shaft of said multifunctional convertible catheter, obtaining radiographic angiogram images, positioning said angioplasty balloon in a location within a blood vessel to be treated using fluoroscopic observation of injected radiopaque contrast material into said guide wire lumen, dilating a vascular lesion using said angioplasty balloon, deflating said angioplasty balloon and repositioning said multifunctional convertible catheter to permit contrast injection to obtain suitable radiographic angiogram images, injecting contrast into a guide wire lumen through a hub adapter, said contrast exiting said guide wire lumen primarily through an intermediate port along a shaft of said multifunctional convertible catheter, and obtaining radiographic angiogram images, and removing devices from said patient's body.

9. A method of performing an angioplasty comprising use of multifunctional convertible catheter comprising a proximal end and a distal end, and said proximal end comprising at least a first hub adapter at a proximal end of said first lumen, and at least a second hub adapter at a proximal end of said second lumen, and at least an expansile balloon securely mounted on said catheter generally disposed toward said distal end of said catheter, said multifunctional convertible catheter comprising a plurality of lumens including at least a first lumen as a means for passage of a guide wire and injection of fluids there through, said first lumen extending continuously from said proximal end to said distal end of said catheter, said first lumen including at least a proximal port at said proximal end of said catheter, at least a distal port at said distal end of said catheter, and at least a third intermediate port located proximal to said expansile balloon of said catheter and distal to said proximal end of said catheter, said intermediate port comprising an opening through a wall of said catheter, said multifunctional convertible catheter further comprising a means for injection of fluids into said first lumen with a guide wire in position throughout said first lumen from said proximal end to said distal end, and said multifunctional convertible catheter further comprising at least a second lumen as a means for injection of pressurized fluids or gases to inflate said expansile balloon, said method of performing an angioplasty comprising a “rapid exchange” (RX) angioplasty by gaining vascular access in a patient using Seldinger's technique or other methods, placing a vascular access sheath in a vascular tree, using a combination of a selective diagnostic catheter and guide wire to selective catheterize an artery of interest, remove said diagnostic catheter while maintaining said guide wire in situ, introducing said multifunctional convertible catheter by “back loading” it over said guide wire, observing guide wire entry into a catheter segment bounded by a third intermediate port, manipulating said multifunctional convertible catheter and said guide wire such that said guide wire passes through said third intermediate port and exterior to said multifunctional convertible catheter shaft, advancing said multifunctional convertible catheter to an appropriate position within a blood vessel to be treated, injecting contrast into a guide wire lumen through a hub adapter said contrast exiting a third intermediate port along a shaft of said multifunctional convertible catheter and obtaining radiographic angiogram images, positioning said multifunctional convertible catheter such that said angioplasty balloon can perform angioplasty in a blood vessel to be treated using fluoroscopic observation of radiopaque contrast injected into said guide wire lumen, dilating a blood vessel in area to be treated using said angioplasty balloon, deflating said angioplasty balloon and repositioning said multifunctional convertible catheter to obtain radiographic angiogram images, injecting contrast into a guide wire lumen through a hub adapter, said contrast exiting said guide wire lumen primarily through a third intermediate port along a shaft of said multifunctional convertible catheter, and obtaining radiographic angiogram images, and removing devices from said patient's body.

10. A method of performing an angioplasty of claim 8 or claim 9, in which the method of claim 8 or of claim 9 is performed using a single catheter.

11. A method of performing an angioplasty of claim 10, with the election of using over-the-wire or rapid-exchange catheter configurations determined by the operator during said single procedure.

12. A method of performing an angioplasty comprising use of a multifunctional convertible catheter comprising a proximal end and a distal end, and at least an expansile balloon securely mounted on said catheter generally disposed toward said distal end of said catheter, said multifunctional convertible catheter comprising a plurality of lumens including at least a first lumen as a means for passage of a guide wire or injection of fluids there through, said first lumen extending continuously from said proximal end to said distal end of said catheter, said first lumen including at least a proximal port at said proximal end of said catheter, at least a distal port at said distal end of said catheter, and at least a third intermediate port located proximal to said expansile balloon of said catheter and distal to said proximal end of said catheter, said intermediate port comprising an opening through a wall of said catheter, and at least a second lumen as a means for injection of pressurized fluids or gases to inflate said expansile balloon, and said proximal end comprising at least a first hub adapter at a proximal end of said first lumen, and at least a second hub adapter at a proximal end of said second lumen, said method comprising performance of diagnostic angiography by radiopaque contrast injection that exits said multifunctional convertible catheter proximal to an expansile balloon generally disposed toward a distal end of said multifunctional convertible catheter, positioning of said expansile balloon within said blood vessel to be treated using fluoroscopic observation of contrast injected via said guide wire lumen, inflation of said expansile balloon, said method further comprising optional conversion of said multifunctional convertible catheter from over-the-wire to rapid-exchange conversion at the election of an operator during performance of said angioplasty procedure.

13. A method of performing balloon angioplasty using an angioplasty balloon catheter with a contrast injection port proximal to an angioplasty balloon, said angioplasty balloon catheter converted between over-the-wire and rapid-exchange configurations.
